(19) United States
(12) Patent Application Publication
(10) Pub. No.: US 2007/0237726 A1
(43) Pub. Date: Oct. 11, 2007

(54) Oral care regimens and kits

(75) Inventors: Donald James White, Fairfield, OH (US); Aaron Reed Biesbrock, Maineville, OH (US); Malgorzata Klukowska, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/732,927

(22) Filed: Apr. 5, 2007

Related U.S. Application Data
(60) Provisional application No. 60/790,505, filed on Apr. 7, 2006.

(51) Int. Cl. A61K 8/19; A61K 8/21; A61K 8/31; A61K 8/34

(52) U.S. Cl. 424/49; 424/52; 424/55; 433/84

(57) ABSTRACT

Disclosed are oral hygiene regimens comprising a plurality of steps involving the use of two or more oral care products containing actives effective to provide one or more benefits including cleaning, antimicrobial, antiplaque, anticaries, anti-calculus, anti-demineralizing, anti-erosion, antisensitivity/desensitizing, whitening, surface conditioning, polishing, and pH-balancing, wherein the plurality of steps are conducted and sequenced for maximizing delivery of oral care actives to target surfaces of a subject's oral cavity thereby achieving optimum oral health and hygiene benefits. In one aspect, a regimen comprises at least once daily of each of the following steps:
(a) applying an antimicrobial dentifrice to oral cavity surfaces, and
(b) rinsing the oral cavity with an antimicrobial mouthrinse,
   wherein at least one of steps (a) and (b) is conducted prior to retiring at night. Preferably the regimen comprises as step (c) treating interproximal, gingival and subgingival areas of the oral cavity using an interproximal device selected from dental floss, floss pick, dental tape, and proxy brush, wherein step (c) is conducted immediately before or immediately after one or both of steps (a) and (b).

### CROSS REFERENCE TO RELATED APPLICATION

This application claims the benefit of U.S. Provisional Application No. 60/790,505, filed Apr. 7, 2006.

### FIELD OF THE INVENTION

The present invention relates to oral care regimens and kits which can be used to maximize delivery of oral care actives to a subject's oral cavity and thereby achieve optimum oral health, hygiene and cosmetic benefits.

### BACKGROUND

Oral care products such as dentifrice and mouthrinse are routinely used by consumers as part of their oral care hygiene regimens. It is well known that oral care products can provide both therapeutic, hygiene and cosmetic benefits to consumers. Therapeutic benefits include caries prevention which is typically delivered through the use of various fluoride salts; gingivitis prevention by the use of an antimicrobial agent such as triclosan, stannous fluoride, or essential oils; or hypersensitivity control through the use of ingredients such as strontium chloride, stannous fluoride or potassium nitrate. Hygiene and cosmetic benefits provided by oral care products include the control of plaque and calculus formation, removal and prevention of tooth stain, tooth whitening, breath freshening, and overall improvements in mouth feel impression which can be broadly characterized as mouth feel aesthetics. Calculus and plaque along with behavioral and environmental factors lead to formation of dental stains, significantly affecting the aesthetic appearance of teeth. Behavioral and environmental factors that contribute to teeth staining propensity include regular use of coffee, tea, cola or tobacco products, and also the use of certain oral products containing ingredients that promote staining, such as chlorhexidine and metal salts.

Dental plaque is a mixed matrix of bacteria, epithelial cells, leukocytes, macrophages and other oral exudates. Bacteria comprise approximately three-quarters of the plaque matrix. Any given sample of dental plaque could contain as many as 400 different varieties of microorganisms. This mix includes both aerobic and anaerobic bacteria, fungi, and protozoa. Viruses have also been found in samples of dental plaque.

This matrix of organisms and oral exudates continues expanding and coalesces with other plaque growths situated nearby. The bacteria synthesize levans and glucans from sugars found in the oral cavity providing energy for the microorganisms. These glucans, levans, and microorganisms form an adhesive skeleton for the continued proliferation of plaque into what is also referred to as a biofilm, which is tenaciously adherent and difficult to remove.

Dental calculus, or tartar as it is sometimes called, is a deposit which forms on the surfaces of the teeth at the gingival margin. Supragingival calculus appears principally in the areas near the orifices of the salivary ducts; e.g., on the lingual surfaces of the lower anterior teeth and on the buccal surfaces of the upper first and second molars, and on the distal surfaces of the posterior molars. Mature calculus consists of an inorganic portion which is largely calcium phosphate arranged in a hydroxyapatite crystal lattice structure similar to bone, enamel and dentine. An organic portion is also present and consists of desquamated epithelial cells, leukocytes, salivary sediment, food debris and various types of microorganisms. Developing plaque can adhere most easily at relatively irregular surfaces, such as those afforded by calculus. As the mature calculus develops, it becomes visibly white or yellowish in color unless stained or discolored by some extraneous agent, becoming unsightly and undesirable from an aesthetic standpoint.

The failure to retard or stop the proliferation of plaque is detrimental to oral health, leading to dental caries, gingival inflammation, periodontal disease, and ultimately tooth loss. The two most prevalent diseases of the periodontium are plaque-induced gingivitis, a reversible condition, and chronic periodontitis, an irreversible condition that can lead to tooth loss. The role of dental plaque in the development of these diseases has been established in many studies. It is believed that the best approach to manage periodontal diseases is prevention, followed by early detection and treatment. Prevention of periodontal diseases is targeted at the control of dental plaque. Chemical agents with anti-plaque activities such as anticmicrobial agents, have been shown to represent a valuable complement to mechanical plaque control, such as by toothbrushing. Many dentifrices and mouthrinses are thus formulated with antimicrobial agents to provide anti-plaque efficacy and to reduce or prevent gingivitis.

A wide variety of dentifrice and mouthrinse products are available, designed to provide one or more of the above therapeutic and aesthetic benefits. Moreover, numerous other oral care products are available for various conditions or treatments such as manual and power toothbrushes, interproximal devices such as dental floss and pick, and bleaching products such as strips and paint-on gels.

Although satisfactory in many respects, a need remains for further advances and improvements in oral health care, specifically in regimens performed by consumers of oral health care products to maximize the benefits derived from such oral care products.

### SUMMARY OF THE INVENTION

The present invention provides oral hygiene regimens comprising a plurality of steps involving the use of two or more oral care products containing actives effective to provide one or more benefits including cleaning, antimicrobial, antiplaque, anticaries, anti-calculus, anti-demineralizing, anti-erosion, antisensitivity/desensitizing, whitening, surface conditioning, polishing, and pH-balancing, wherein the plurality of steps are conducted and sequenced for maximizing delivery of oral care actives to target surfaces of a subject's oral cavity thereby achieving optimum oral health and hygiene benefits.

In one aspect, a regimen according to the present invention comprises at least once daily of each of the following steps:

(a) applying an antimicrobial dentifrice to oral cavity surfaces, and

(b) rinsing the oral cavity with an antimicrobial mouthrinse.

### Preferably at least one of steps (a) and (b) is conducted prior to retiring at night.

The benefits derived from such a regimen include superior plaque control, breath malodor reduction and anti-gingivitis efficacy. Preferably, each of steps (a) and (b) is conducted at least twice daily. The steps may be conducted concurrently or at spaced intervals, for example at least about 30 minutes between steps. Thus one regimen may include applying an antimicrobial dentifrice by toothbrushing, preferably with a power toothbrush, followed by rinsing with an antimicrobial mouthrinse. Or, the regimen may include first rinsing and then brushing. The steps in the regimen are aimed at removing existing plaque and providing immediate and sustained antimicrobial action in the mouth thereby inhibiting plaque formation or regrowth.

The regimen preferably includes an additional step (c) of cleaning and treating interproximal, gingival and subgingival areas of the oral cavity using an interproximal device selected from dental floss, floss pick, dental tape, and proxy brush, wherein step (c) is conducted immediately before or immediately after one or both of steps (a) and (b).

Preferred antimicrobials contained in the dentifrice and mouthrinse products include a stannous ion source, a zinc ion source, triclosan, a peroxide source, cetyl pyridinium chloride, domiphen bromide, chlorhexidine, triclosan, triclosan monophosphate, essential oils and mixtures thereof.

These and other features, aspects, and advantages of the present invention will become evident to those skilled in the art from the detailed description which follows.

### DETAILED DESCRIPTION OF THE INVENTION

While the specification concludes with claims particularly pointing out and distinctly claiming the invention, it is believed that the present invention will be better understood from the following description.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

Herein, “comprising” means that other steps and other components which do not affect the end result can be added. This term encompasses the terms “consisting of” and “consisting essentially of.”

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

By “oral care composition” is meant a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care compositions of the present invention may be in various forms including dentifrice, toothpaste, tooth gel, mousse, foam, subgingival gel, mouthrinse or mouthwash, mouthspray, lozenge, chewable tablet or chewing gum. The oral care composition may also be incorporated onto strips or films for direct application or attachment to oral surfaces.

The term “dentifrice”, as used herein, means paste, gel, serum, concentrate or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side. The dentifrice may be applied to teeth, gums and other oral surfaces by brushing, by painting onto the surface or by adhering a strip coated with the dentifrice composition onto the oral surface.

The term “orally acceptable carrier or excipients” includes safe and effective materials and conventional additives used in oral care compositions including but not limited to fluoride ion sources, anti-calculus or anti-tartar agents, buffers, abrasives such as silica, alkali metal bicarbonate salts, thickening materials, humectants, water, surfactants, titanium dioxide, flavor system, sweetening agents, xylitol, coloring agents, and mixtures thereof. The choice of a carrier to be used is basically determined by the way the composition is to be introduced into the oral cavity. Carriers suitable for the preparation of compositions of the present invention are well known in the art. Their selection will depend on secondary considerations like taste, cost, and shelf stability, etc. Carrier materials for various types or oral care compositions are disclosed in e.g., U.S. Pat. No. 3,988,433 to Benedict; U.S. Pat. No. 4,083,955, to Grabenstetter et al.; U.S. Pat. No. 5,198,220 and U.S. Pt. No. 5,242,910, both to Damani; U.S. Pat. Nos. 5,213,790, 5,145,666, and 5,281,410 all to Lukacovic et al.; U.S. Pat. Nos. 4,849,213 and 4,528,180 to Schaeffer; U.S. Pat. No. 5,939,052 to White, et al.; U.S. Pat No. 6,696,045 to Yue et al.; U.S. Pat. No. 6,740,311 to White et al.; U.S. Pat. No. 6,846,478 to Doyle, et al. and U.S. Pat. No. 7,063,833 to Glandorf, et al.

Active and other ingredients useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

Herein, the term “biofilm” refers to aged dental plaque. The terms “tartar” and “calculus” are used interchangeably and refer to mineralized dental plaque biofilms.

In one embodiment, the regimen comprises brushing with a stannous-containing dentifrice followed by rinsing with a mouthrinse comprising high bioavailable quaternary ammonium antimicrobials such as cetyl pyridinium chloride (CPC), such as described in commonly-assigned U.S. Application No. 2005000037560, published as US 20050169852A1 on Aug. 4, 2005. Suitable stannous dentifrice formulations include those disclosed in commonly-assigned U.S. Pat. Nos. 5,004,597; 5,939,052; 6,187,295; 6,350,436, 6,667,027; 6,521,216, 6,555,094; 6,696,045; 6,821,507; 6,713,049; and 6,685,920 and in U.S. Pat. Nos. 5,871,715; 5,017,363 and 5,009,883, assigned to Gillette.

In one embodiment the regimen includes step (a) brushing teeth and oral cavity surfaces using a toothbrush with a dentifrice comprising

(i) a stannous ion source in an effective antimicrobial amount,

(ii) a fluoride ion source in an effective anticaries amount, and

(iii) one or more linear polyphosphates having an average chain length of about 4 or more.

The toothbrush may be manual, battery-powered or electric kind. However, in clinical studies it has been demonstrated that power brushes provide a larger benefit than manual brushes, and may be preferred in the present regimens. Examples of suitable toothbrushes include those manufactured by Oral-B® under the brand names Pulsar Pro-Health™, Cross Action and Renewal Daily Whitening.

Step (a) is followed by step (b), which includes rinsing teeth and oral cavity surfaces with a mouthrinse comprising

(i) one or a mixture of quaternary ammonium antimicrobial agents in an amount to deliver at least about 300 ppm bioavailable quaternary ammonium antimicrobial agent selected from the group consisting of cetylpyridinium chloride (CPC), tetradecylpyridinium chloride, N-tetradecyl-4-ethyl pyridinium chloride, domiphen bromide, and mixtures thereof, and

(ii) a pharmaceutically-acceptable liquid carrier comprising a major proportion of water and from about 5% to about 30% by weight of the composition of a polyhydric alcohol humectant.

Preferably, the mouthrinse comprises at least about 0.035% cetylpyridinium chloride (CPC) by weight of the composition. The rinsing step may be conducted immediately before or after the brushing step or may be conducted after at least about 30 minutes has elapsed after brushing. In clinical studies, it has been demonstrated that larger additive benefits may be derived when spacing the brushing and rinsing steps in terms of reduction of plaque regrowth and breath malodour. It is believed brushing with an effective antimicrobial dentifrice may sufficiently remove most of the plaque bacteria and immediately rinsing with an antimicrobial rinse may provide only a small incremental benefit. The benefit may be of a larger magnitude if the rinsing step is conducted after some time has elapsed, i.e., when plaque has started reforming. In addition, there may be ingredients from the dentifrice left in the mouth that may interfere with the antimicrobial active in the rinse. For example, anionic surfactants and additives in the dentifrice may interfere with the bioavailability of cationic antimicrobials such as CPC present in the mouthrinse. An example of a daily regimen includes brushing and rinsing in the morning, rinsing after lunch and after dinner and brushing and rinsing at night prior to retiring.

The regimen will preferably further include the step of flossing, preferably immediately before or immediately after the brushing and rinsing steps. The flossing step cleans the areas between the teeth, the gum line and other hard to reach areas and makes these areas more accessible for delivery of actives from the dentifrice and rinse. Preferably, the floss itself contains an antimicrobial active that is delivered during flossing. The floss may be supplied already containing an antimicrobial or the consumer may impregnate the floss with the antimicrobial dentifrice or mouthrinse as part of the regimen.

The regimen will preferably also include a plaque disclosing means for identification, location and quantification of plaque deposits in the oral cavity to assist in performing the necessary treatment steps such as brushing, flossing and rinsing, to remove such plaque. Plaque disclosing products generally contain coloring agents or pigments that are absorbed by the plaque and render it visible. Most plaque disclosing compositions are based on colorants such as disclosed in U.S. Pat. Nos. 3,309,274; 3,624,219; 3,997,658; 4,302,439; 4,459,277; 4,517,172; 4,590,061; 4,666,700; 4,992,256; 5,098,691; 5,190,743; 7,182,935. Examples include synthetic organic colorants such as, amongst others, erythrosin (FD&C Red #3), Allura Red (FD&C Red #40), Green #8, Red #19, Red #22, Red #28, fluorescein (Yellow #7) and fluorescein disodium salt (Yellow #8). Natural colorants that have been used include a red dye extracted from sugar beet, a salt of sanguinarine, and cobalamin compounds, particularly cyanobalamin (Vitamin B12). Some of these colorants are invisible to the human eye in normal daylight or artificial light and may require the use of light of a particular wavelength to become visible. The plaque-disclosing agent may be incorporated in the dentifrice, rinse or interproximal device or may be provided as a separate product in various forms such as tablets, solutions, gels or aerosols.

Further, the regimen may include a disinfecting/sanitizing step using the antimicrobial mouthrinse as disinfectant for the toothbrush or interproximal device to avoid reintroduction of microbes in the mouth. The brush or device may be soaked in mouthrinse after use, preferably overnight.

The present invention also provides oral hygiene kits to assist consumers in complying with a recommended regimen. One example of a kit comprises an antimicrobial dentifrice (e.g., Crest® PRO-HEALTH™ stannous fluoride dentifrice) for use in combination with a toothbrush (e.g., Oral-B® Pulsar Pro-Health™ brush); an antimicrobial mouthrinse (e.g., Crest® PRO-HEALTH™ CPC mouthrinse); at least one interproximal device (e.g., Oral-B® Satin floss) and instructions for conducting a regimen to achieve optimum benefits. Such a kit and regimen would be particularly useful for consumers having or at risk for development of gingivitis and periodontal disease, for example, consumers that experience bleeding and sore gums and those diagnosed or indicated to suffer from significant gingival detachment, i.e., 3 mm or greater. Such consumers are also at risk for development of a number of systemic diseases.

It is now well established that oral infections can lead to systemic infection. Bacteria can spread from the mouth into the bloodstream and other parts of the body, thereby putting a person's health at risk. Recent research has found that periodontal infection may contribute to the development of a number of serious conditions including heart disease, diabetes, respiratory diseases and premature, underweight births. Chronic periodontal infection has been shown to produce a biologic burden of bacterial toxins and inflammatory cytokines that may initiate and exacerbate atherosclerosis and thromboembolic events. Additionally, a known periodontal pathogen, Porphyromonas gingivalis has been isolated from arteriosclerotic plaques. Periodontal disease has also been shown to induce episodes of significant bacteremias and thromboembolic events such as myocardial infarction and stroke can occur following bacteremia. Bacteria associated with periodontal disease such as Streptococcus sanguis and Porphyromonas gingivalis, have been demonstrated to cause platelets to aggregate upon contact with these bacteria. The resultant bacterially-induced platelet aggregates can form the emboli which are responsible for the acute myocardial infarction or stroke.

The present regimens that provide enhanced antimicrobial efficacy in treating and preventing oral infections are also beneficial toward promoting systemic health. Antimicrobial compositions that may be used in the present regimens for treating diseases and infections of the oral cavity and for promoting systemic or whole body health are disclosed in commonly assigned U.S. Pat. Nos. 6,846,478 and 6,696,045 and applications published as US 2003/0206874A1, US 2005/0163727A1, US 2005/0169852A1, and WO 02/02096. Specifically, spread into the bloodstream of pathogenic oral bacteria, associated bacterial toxins and endotoxins, and resultant inflammatory cytokines and mediators prompted by these oral pathogens is prevented or minimized, thereby decreasing etiologic factors that contribute to development of systemic diseases, such as heart disease in humans and in other animals. By decreasing the etiologic factors for a systemic disease, the risk of developing such a disease is also decreased leading to better overall systemic health for the subject.

The effectiveness of the present regimens is demonstrated in the following clinical tests.

### Regimens for Treatment of Oral Malodor

In this study, the efficacy of combining product forms containing antimicrobial agents (dentifrice and mouthrinse) into a systematic oral care hygiene regimen was assessed relative to the use of just one form alone (antimicrobial dentifrice or mouthrinse). Improved breath status as the desired oral outcome was used to evaluate the following regimens (A-F).
A. Sodium Fluoride dentifrice [Crest® Cavity Protection (CCP) dentifrice]
B. Stannous fluoride dentifrice [Crest® PRO-HEALTH™ dentifrice (CPHD)]
C. Triclosan dentifrice+Essential oils mouthrinse [Colgate Total® dentifrice+Listerine® mouthrinse, manufactured by Colgate-Palmolive and Pfizer, Inc., respectively]
D. Sodium Fluoride dentifrice+Cetylpyridinium Chloride (CPC) mouthrinse [Crest® Cavity Protection (CCP) dentifrice+Crest® PRO-HEALTH™ mouthrinse (CPHR), manufactured by The Procter & Gamble Co.]
E. Stannous fluoride dentifrice+Cetylpyridinium Chloride (CPC) mouthrinse [Crest® PRO-HEALTH™ dentifrice (CPHD)+Crest® PRO-HEALTH™ mouthrinse (CPHR), manufactured by The Procter & Gamble Co., morning and evening usage]
F. Stannous fluoride dentifrice+Cetylpyridinium Chloride (CPC) mouthrinse [Crest® PRO-HEALTH™ dentifrice (CPHD)+Crest® PRO-HEALTH™ mouthrinse (CPHR), morning and evening dentifrice usage, mouthrinse usage after lunch and after dinner]

The clinical was a single-center, examiner blinded, 6-treatment, 6-period, open label, cross-over study. Thirty subjects who had shown evidence of reproducible malodor based on a screening exercise conducted outside of this protocol were enrolled in this study. Breath measurements were taken at Baseline and approximately 24 hours and 48 hours post-baseline of each treatment period. Halimeter measurements were taken at each study visit. Measurements were taken in the morning before any oral hygiene was conducted. Subjects were assessed for volatile sulfur compound (VSC) emissions utilizing a commercially-available portable instrument called a Halimeter (Interscan Corporation, CA). This instrument is sensitive to hydrogen sulfide and methyl mercaptan, two of the primary components of foul breath odor. A trained technician performed all Halimeter measurements. Calibration of the Halimeter was performed according to procedures described by manufacturer.

Subjects were given acclimation products of dentifrice (Crest® Cavity Protection) and brush (ADA manual reference) to be used in the morning and evening approximately 7 days prior to their first Baseline Visit and during washout between treatment periods.

During acclimation, subjects were instructed to dispense a full stripe of dentifrice and brush twice daily (once in the morning and once in the evening) in their customary manner for 60 seconds, excluding brushing of the tongue, followed by a dentifrice slurry swish for 20 seconds. Subjects then rinsed their mouths out twice with 15 mL of water for 10 seconds each time.

During the treatment period, subjects brushed their teeth in the same manner as during the acclimation period. If on a regimen with a mouthrinse, subjects rinsed using 20 mL of their assigned mouthrinse for 30 seconds and expectorated. Three regimens including mouthrinse product instructed the subjects to use the assigned rinse immediately following brushing in the morning and evening and one regimen instructed the subjects to use the assigned rinse after lunch and dinner. Subjects were instructed to refrain from eating and drinking for 30 minutes after their product(s) usage.

At the Baseline Visit, subjects were asked to provide samples for a breath measure after not having performed any oral hygiene, eaten, or drank anything from the night before. Once the Halimeter measure had been taken, subjects were randomly assigned to one of the 6 test regimens and then performed a supervised usage of their assigned product(s). Subjects used their assigned treatment regimen that evening and two times the next day for a total of 4 product usages.

The subjects returned after their 2nd product usage for a repeat of the breath measure taken at the Baseline Visit (approximately 24 hour post-baseline). Subjects were again measured for breath approximately 48 hours after the Baseline Visit. This procedure was repeated for each of the 6 treatment periods. Results are summarized in Table 1 below.

The average baseline VSC score for the study was approximately 122.0 ppb. After 24 and 48 hours of treatment these scores dropped to adjusted means levels ranging from 72.6 ppb to 118.1 ppb, depending on the treatment regimen. At both the 24 hour and 48 hour visits the adjusted mean VSC level of the Crest Cavity Protection treatment regimen was significantly (p≦0.0825) the highest. Each of the antimicrobial regimens (C, E and F) had significantly or directionally lower VSC scores with Regimen F (stannous dentifrice+CPC mouthrinse After Meals treatment regimen) having the lowest adjusted mean VSC score. The relative rank ordering of the other treatment regimens varied from Hour 24 to Hour 48.

This study demonstrates that the regimen approach of combining product forms provides greater breath benefits than dentifrice and mouthrinse products used separately. Regimens combining an antimicrobial dentifrice (stannous fluoride dentifrice or triclosan dentifrice) with an antimicrobial mouthrinse (high bioavailable CPC mouthrinse or essential oils mouthrinse) provide superior breath odor benefits, measured in the morning. There is some evidence to suggest that staggering the use of treatment forms over the day may be a better regimen approach than coupling brushing and rinsing together. There were no reported adverse events throughout the study.

TABLE 1
TREATMENT COMPARISONS - ANALYSIS OF COVARIANCE FOR
CROSSOVER DESIGNS VOLATILE SULFUR COMPOUNDS (PPB)^{a}
EVALUABLE SUBJECTS

| TREATMENT | | ADJUSTED | TREATMENT COMPARISON P-VALUES^{c} | | | | |
|---|---|---|---|---|---|---|---|
| GROUP | N^{b} | MEAN (SE) | B | D | C | E | F |

24 HOUR (baseline mean = 122.0, between-subject variance = 0.17, error
variance = 0.09)

| | | | | | | | |
|---|---|---|---|---|---|---|---|
| A | 29 | 118.1 (0.10) | 0.0081 | 0.0111 | 0.0004 | 0.0005 | <0.0001 |
| B | 30 | 97.2 (0.09) | | 0.9310 | 0.2973 | 0.1455 | 0.0003 |
| D | 29 | 97.8 (0.10) | | | 0.2624 | 0.2607 | 0.0005 |
| C | 28 | 89.2 (0.10) | | | | 0.4862 | 0.0080 |
| E | 26 | 89.0 (0.10) | | | | | 0.0183 |
| F | 27 | 72.6 (0.10) | | | | | |

48 HOUR (baseline mean = 120.7, between-subject variance = 0.07, error
variance = 0.12)

| | | | | | | | |
|---|---|---|---|---|---|---|---|
| A | 29 | 110.6 (0.08) | 0.0825 | 0.0373 | 0.0003 | 0.0028 | 0.0002 |
| B | 29 | 97.2 (0.08) | | 0.6619 | 0.0306 | 0.0718 | 0.0120 |
| D | 27 | 93.2 (0.08) | | | 0.0895 | 0.3120 | 0.0739 |
| C | 28 | 79.2 (0.08) | | | | 0.7447 | 0.4425 |
| E | 26 | 84.4 (0.08) | | | | | 0.4299 |
| F | 26 | 78.0 (0.09) | | | | | |

^{a}Data were analyzed on the natural logarithm scale. Means were transformed back to original scale for reporting.
^{b}The number of subjects receiving the given treatment.
^{c}Bold-faced treatment comparison p-values are one-sided in the direction of greater efficacy for the column treatment.

### Regimens for Control of Plaque Regrowth

This study demonstrated the plaque regrowth inhibition benefits from the present regimens. Control of bacterial plaque in the mouth is essential to preventing oral cavity conditions including caries, gingivitis, periodontal disease, and tooth loss. Mechanical tooth cleaning practices (toothbrushing, flossing, rinsing) in combination with chemical agents (antimicrobials or agents which prevent bacterial attachment to surfaces or detach plaque) are used to control plaque. The general correlation between plaque growth and vitality and gingivitis susceptibility suggests that an assessment of antiplaque properties of a regimen—particularly throughout the day, would be ideal to dimensionalize the potential combined effect and duration of therapeutic activity. The methodology used for the assessment of plaque cleaning or removal and inhibition of plaque formation or regrowth is the DPIARM technique (Repeated Measures Digital Plaque Image Analysis) described in White et al. J Clin Dent 17:22-26, 2006.

Previous testing of CPC formulations and Essential Oil mouthrinse formulations with this method indicated that the rinses used twice daily after brushing, according to instructions, had a large significant effect on diurnal plaque at all time points measured. Likewise, previous separate testing of dentifrices containing stannous fluoride or triclosan with digital plaque imaging provided evidence that these formulations also affect diurnal plaque to a lesser extent. The DPIARM technique is thus used herein to test the effect of a combined regimen of antimicrobial rinses and dentifrices on overall antiplaque efficacy.

This study employed a treatment intervention design to examine the combined effect of a regimen of 700 ppm CPC mouthrinse (Crest® PRO-HEALTH™ mouthrinse) used twice daily, morning and night, after brushing with a stannous fluoride dentifrice (Crest® PRO-HEALTH™ dentifrice) versus the combined effect of a regimen of an Essential Oils mouthrinse (Listerine) used twice daily, morning and night, after brushing with a triclosan dentifrice (Colgate Total®), and versus the baseline (Crest Cavity Protection dentifrice only) and versus the stannous fluoride dentifrice only or versus the triclosan dentifrice only on diurnal plaque level response in the DPIARM panel.

Diurnal plaque formation levels observed during use of Crest Cavity Protection (CCP) dentifrice were used to verify equilibrium plaque levels in a pre study period to the baseline (period A) of the study. Once equilibrium plaque levels have been verified in panelists, one week of baseline CCP plaque response were recorded. Following this, the panel was split into two groups balancing for baseline average plaque levels, and diurnal plaque levels were assessed during one week with half of panel using Crest® PRO-HEALTH™ stannous fluoride dentifrice twice daily in place of CCP and half of panel using Colgate Total triclosan dentifrice twice daily in place of CCP. This period was aimed at measuring efficacy of antimicrobial dentifrices. The study then continued for an additional 3 weeks with the addition of twice daily use of antimicrobial mouthrinse. Panelists using the Crest® PRO-HEALTH™ stannous fluoride dentifrice added Crest® PRO-HEALTH™ rinse, and panelists using Colgate Total® dentifrice added Listerine® rinse.

Dental plaque levels were evaluated using standardized Digital Plaque Image Analysis protocol to disclose the total area of tooth (in pixels) and total area covered with plaque. Total tooth pixel is used to cross check precision of the repositioning and assessment. The % of teeth covered with plaque following each disclosure is measured for each treatment. This is derived from measurements of tooth surface covered with plaque and total tooth surface (plaque free+plaque-covered).

Plaque disclosure for imaging utilized a fluorescein buffer solution containing 1240 ppm fluorescein. Prior to photographing, subject plaque is disclosed by fluorescein using the following procedure:

Rinse for 10 seconds with 25 ml of phosphate buffer;

Rinse for 1 minute with 5.0 ml of 1240 ppm fluorescein in phosphate buffer;

Rinse 3× for 10 seconds with 25 ml of phosphate buffer.

Phosphate buffer is comprised of 3.62 grams of monosodium phosphate and 0.349 grams of disodium phosphate diluted to 2 liters with ultrapure water. The final pH of this mixture is 5.5. Solution is prepared fresh—in a GMP approved process laboratory each day.

For each treatment period, subjects were provided with a test dentifrice and an Oral-B® 40 Soft toothbrush and instructed to brush as they normally do twice per day with the evening brushing taking place right before they retire for the evening. During treatment periods involving mouthrinse, rinsing followed toothbrushing—both morning and evening. Toothbrushing was followed with extra water rinses to wash out any residual surfactant from the dentifrice. Following brushing and water rinsing, subjects were instructed to dispense roughly 20 ml of mouthrinse into their assigned dose cup, to rinse for 30 seconds (timer provided) and to expectorate the mouthrinse. Following evening rinsing they were instructed not to rinse with further water, eat or drink prior to retiring for the evening. In the morning, mouthrinse use will likewise follow morning toothbrushing, though rinse use will differ on graded vs. non graded days. On non graded days, subjects were instructed to rinse with their assigned mouthrinse following a.m. toothbrushing at home, and again allowing for extra rinsing of water between brushing and rinse applications. In morning use, subjects were instructed not to eat or drink for 30 minutes following rinse applications. On grading days, a different morning use was followed.

Each week, subjects were graded on 3 days with 3 measurements each day: (1) pre-brush a.m., (2) post-brush a.m. and (3) p.m. Subjects reported to the imaging laboratory for grading in the morning prior to any food/beverages and without further oral hygiene. (Each subject would have brushed as usual and rinsed with assigned product the evening before). Subjects then disclosed dental plaque (using a fluorescein rinse) and subjected themselves to ‘pre brush a.m.’ plaque imaging, followed by a timed brushing for 40 seconds with assigned dentifrice provided in metered 1.5 gram doses using an Anchor™ disposable flat-head brush. Following brushing, subjects redisclosed dental plaque and subjected themselves to a second plaque imaging (post brush a.m. plaque imaging). Following the post brush plaque grading, subjects were asked to rinse. 3× more with phosphate buffer rinse solution and 3× more with water to wash out any residual fluorescein dye. Subjects then rinsed with assigned mouthrinse and were asked to refrain from eating/drinking (no coffee etc.) for 30 minutes further. Following this, subjects were free to have breakfast and lunch, as well as snacks etc. throughout the grading day. In the afternoon (from about 2-3:00 p.m.) subjects again reported to the dental imaging lab for a third plaque disclosure and measurement. Subjects were instructed to avoid food and drink for at least ½ hour prior to this evaluation. The antimicrobial dentifrice+rinse treatment period occurred over three weeks providing 9 repeat measures of plaque formation. Results of this study summarized below demonstrate large additive reductions from a regimen combining an antimicrobial dentifrice and antimicrobial rinse, with the most plaque reduction and plaque regrowth inhibition derived from the stannous fluoride dentifrice and high bioavailable CPC rinse combination. This regimen provided the least plaque regrowth during the day, akin to a “just brushed” condition over a long period of time. Plaque levels throughout a 24-hour period remained constantly low, thereby providing protection from development of plaque-induced oral cavity diseases.

| | | Mean % Plaque on Teeth Measured via DPIA | | | |
|---|---|---|---|---|---|
| | | a.m. pre- | a.m. post | | % Reduction |
| Treatment | N | brush | brush | p.m. | vs. CCP (p.m.) |
| CCP (Group 1) | 9 | 13.80 | 6.92 | 14.23 | |
| CCP (Group 2) | 7 | 15.46 | 8.04 | 12.93 | |
| CPH dentifrice | 9 | 11.30 | 6.52 | 10.32 | 27.48 |
| Colgate Total ® dentifrice | 7 | 15.85 | 8.63 | 13.10 | −1.31 |
| Week 3 | | | | | |
| CPH dentifrice + rinse | 9 | 7.18 | 5.55 | 6.12 | 56.98 |
| Colgate Total ® + Listerine ® | 7 | 8.90 | 6.70 | 8.12 | 37.17 |
| Week 4 | | | | | |
| CPH dentifrice + rinse | 9 | 5.64 | 4.48 | 5.05 | 64.48 |
| Colgate Total ® + Listerine ® | 7 | 7.67 | 5.83 | 6.94 | 46.31 |
| Week 5 | | | | | |
| CPH dentifrice + rinse | 9 | 4.97 | 4.20 | 4.31 | 69.68 |
| Colgate Total ® + Listerine ® | 7 | 7.38 | 5.82 | 6.81 | 47.30 |

### Power Toothbrush Study

In this study the additive effectiveness of an antimicrobial dentifrice (stannous fluoride+hexametaphosphate, Crest® PRO-HEALTH™, CPHD) and a power toothbrush (Oral-B® Triumph™, OBT) was assessed in an intervention based Digital Plaque Image Analysis methodology (DPIA). Sixteen subjects were assigned commercial tubes of Crest® Cavity Protection dentifrice (CCP) and Oral-B® Triumph™ Professional Care 9000™ toothbrushes (CCP-OBT) with instructions for bid brushing morning and evening. Subjects remained on the CCP-OBT regimen for two weeks. During week 2, subjects were evaluated for diurnal plaque levels in 3 separate grading days each including assessments of pre brush a.m.; post brush a.m. and p.m. (mid afternoon) plaque regrowth respectively using standardized UV imaging techniques as described previously (White et al. J Clin Dent 17:22-26, 2006). At week 3, subjects replaced the CCP dentifrice with an antimicrobial stannous fluoride dentifrice (CPHD) and subjects continued brushing for two additional weeks, with plaque re-evaluated during week 4.

Pre brushing (mean plaque % ±SD): CCP: 8.8±4.9; CPHD=6.3±4.2 (29.1% relative reduction p<0.05); Post brushing: CCP: 2.6±1.8; CPHD=2.1±1.3 (17.9% relative reduction, not significant); p.m. regrowth: CCP: 5.6±3.0; CPHD=4.1±2.5 (26.8% relative reduction p<0.05). These results demonstrate that the application of a clinically proven antimicrobial stannous fluoride dentifrice further improved the clinically significant oral hygiene effectiveness of a power toothbrush (OBT), primarily by controlling plaque regrowth between hygiene interventions. CPHD provides additive therapeutic effectiveness in power brush users. Power brushes are therefore preferred in the present regimens and kits.

Regimens may be designed for daily, bi-weekly, weekly, monthly, or any other time period. A regimen may be designed for maximum benefit if it is performed at certain times of the day such as at night, in the morning, within a certain time period (for example over four hours), or throughout the day. A weekly regimen may include the use of one or more products that are only used once or twice per week. A whitening product may only be used once a week, another day may be for use of a deep cleaning dentifrice, and another day for use of an intensive product. The intensive product may be a gel, serum or other form that provides extra fluoride, enhanced antimicrobials or any other oral care active ingredient that provides a benefit from use on a less than daily basis.

One step in a regimen may comprise the use of an activator composition. The activator composition may be a rinse or gel or in any other form that delivers the composition to the oral surfaces. The activator composition is intended to enhance the treatment or effect of the subsequent step. For example, an activator rinse may be used pre-brushing to enable better fluoride absorption during brushing with a fluoride dentifrice. An activator gel may be used as a pre-whitening step for better whitening or peroxide absorption.

Another embodiment contemplates an intensive night treatment to protect the mouth throughout the night when the mouth is most vulnerable for plaque bacteria to flourish, as evidenced in a common consumer complaint of morning mouth malodor. The regimen includes a rinsing step using an activator rinse followed by application of a treatment product containing ingredients such as whitening agents, antimicrobials, and fluoride. The intensive treatment product preferably will include as carrier for the oral care active(s), a material that is substantive to teeth and other oral surfaces and will thus deposit a coating thereon to facilitate deposition and retention of actives onto the oral surfaces where they can perform their intended function. In addition, the substantive coating provides resistance to soiling, staining and adherence of bacteria and other unwanted deposits. Compositions suitable as intensive treatment products are disclosed for example in U.S. Pat. No. 7,025,950 and U.S. application Ser. No. 10/430,520 published as US 20030211050A1 using anionic functionalized polysiloxanes as substantive agent and in U.S. Pat. Nos. 6,555,094; 6,821,507 and 6,713,049 using polyphosphates.

One step in a regimen may comprise a booster product. This may be a composition which is put on the toothbrush with a dentifrice. The booster product may be a serum, gel, liquid, powder or other form that could be combined with a dentifrice. The booster product may be used occasionally with a brushing step or as specified in a regimen.

In another embodiment, a regimen is designed for balancing and controlling the pH in the oral cavity. The regimen includes the steps of brushing and rinsing with an antimicrobial product. The antimicrobial products may preferably be formulated to provide enhanced buffering capability in the mouth. The steps in the regimens are preferably spaced apart for maximum effectiveness. Preferably, a rinsing step will occur at least 30 minutes after, at least 60 minutes after and up to 120 minutes after brushing. The regimen also preferably comprises a rinsing or brushing step after each meal. A kit for a regimen for balancing the pH in the oral cavity may include an antimicrobial dentifrice, an antimicrobial mouthrinse, and a small or travel size antimicrobial dentifrice or mouthrinse for use away from home.

As described above, the present regimens may include use of interproximal devices such as dental floss as disclosed for example, in U.S. Pat. No. 5,518,012 to Dolan et al., which discloses an expanded polytetraflouroethylene (PTFE) floss that can incorporate antimicrobial agents such as cetyl pyridinium chloride (CPC). A dental floss containing a first antimicrobial agent may be used after a rinse also containing the first antimicrobial agent and/or a second antimicrobial agent. For example, a dental floss could contain CPC and the rinse could also contain CPC or, alternatively, hydrogen peroxide. A rinse containing high bioavailable levels of CPC is marketed by the Procter & Gamble Company as Crest® PRO-HEALTH™. The rinse and dental floss might be used in the evening in combination with a strip of material containing a peroxide active which might be used in the morning or anytime prior to evening. An example of such as strip of material is disclosed in U.S. Pat. No. 5,891,453 to Sagel et al., which might be used in the morning. Alternatively, the strip of material could contain an anti-microbial or anti-bacterial agent such as disclosed in U.S. Pat. No. 6,096,328 to Sagel et al. In yet another embodiment, the strip of material can contain a tooth whitening agent in combination with one or more an anti-microbial agents, an example of which is disclosed in U.S. Application No. 60/701,778 filed Jul. 22, 2005 entitled Tooth Whitening Products. In yet another embodiment, a rinse and floss containing an antimicrobial agent can be used in the evening in combination with a strip of material containing an anti-microbial agent that can be worn while sleeping, a strip of material that could be suitable for use while sleeping and which could incorporate an antimicrobial agent is disclosed in U.S. Pat. No. 6,649,147 to Ye et al. The foregoing regimens can further be combined, in whole or part, with a toothbrush that can deliver an antimicrobial agent to the oral cavity or which can prevent or reduce the growth of microbes on a toothbrush and thereby reduce or eliminate transmission of microbes from a toothbrush to the oral cavity, examples of which are disclosed in U.S. Pat. Nos. 5,998,431 and 6,009,589. Any of the foregoing products can be combined and packaged as a kit and distributed as a single system of oral care components.

In yet another embodiment, a toothbrush that delivers oxygen or oxygen radicals at or below the gingival tissues can be combined in whole or part with the regimens and products described above. In one example, a vibrating toothbrush can be used to deliver oxygen or oxygen radicals to the gingival tissue. A toothbrush that could be suitable for use is disclosed in U.S. Pat. No. 5,378,153. A toothbrush that delivers a composition comprising an oxygen generating agent, such as a peroxide (e.g., hydrogen peroxide, carbamide peroxide, and calcium peroxide), to the gingival tissue can also be used. Examples are disclosed in U.S. Pat. Nos. 5,476,384 and 6,648,641 of toothbrushes that could dispense and deliver a composition comprising an oxygen generating agent to, or below the gingival tissue. In one regimen, a rinse or floss comprising an oxygen generating agent might be used in combination with a toothbrush that dispenses or delivers an oxygen generating agent. In another embodiment, a rinse or floss that delivers a first agent to, or below, the gingival tissue might be used in combination with a toothbrush that delivers a second agent that, when combined with the first agent, generates oxygen, oxygen radicals, other radicals, and/or mixtures thereof. Alternatively, the toothbrush might deliver the first agent and the rinse and/or floss might deliver the second agent. The first agent might be provided with an affinity for tartar, plaque, or oral tissues (e.g., soft and/or hard tissues) so that application of the second agent generates oxygen, oxygen radicals, or other radicals at the locations where bacteria and other microbials may be concentrated, including locations at or below the gingival tissue. In yet another embodiment, the floss might deliver the first agent and the rinse might deliver the second agent. Examples of compositions that can adhere to oral/organic tissues to deliver a first agent are disclosed in U.S. Publication Nos. 2003/0211051 and 2003/0211050. First and second agents that can generate oxygen, oxygen radicals, other radicals, and/or mixtures thereof, directly or indirectly, that might be suitable for use are disclosed for example in U.S. Pat. No. 5,302,375 to Viscio.

In still another embodiment, a toothbrush having a light emitting element that can interact with a dental floss, rinse, or dentifrice is combined with the compositions, products, steps and regimens described herein. An example of such toothbrush is disclosed in U.S. Application No. 60/774,710.

The following non-limiting examples further describe mouthrinses and dentifrices suitable for use in the present regimens. All percentages used herein are by weight of the composition unless otherwise indicated. These compositions may be made using conventional methods.

### Antimicrobial Mouthrinse Compositions

| Component | Ex. 7 | Ex. 8 | Ex. 9 | Ex. 10 | Ex. 11 | Ex. 12 | Ex. 13 | Ex. 14 |
|---|---|---|---|---|---|---|---|---|
| Cetylpyridinium Chloride | 0.040 | 0.075 | 0.070 | 0.050 | 0.045 | 0.075 | — | — |
| Domiphen Bromide | — | — | — | — | 0.005 | — | — | — |
| Zinc Lactate | — | — | — | 0.250 | — | 0.050 | — | — |
| Hydrogen Peroxide¹ | — | 2.143 | — | — | — | — | 4.286 | 4.286 |
| Glycerin | 23.000 | 20.000 | 20.000 | 13.000 | 5.000 | 18.000 | 11.000 | 11.000 |
| Propylene Glycol | | 4.000 | | | | | 3.000 | — |
| Na Polyphosphate² | | — | | | | — | 1.00 | 1.00 |
| Flavor | 0.080 | 0.210 | 0.120 | 0.160 | 0.080 | 0.190 | 0.205 | 0.205 |
| Sweetener³ | 0.025 | 0.060 | 0.018 | 0.030 | 0.025 | 0.020 | 0.080 | 0.068 |
| Poloxamer 407 | — | 0.100 | 0.050 | 0.025 | — | 0.001 | 0.750 | 0.750 |
| Monosodium Phosphate | 0.085 | — | — | — | — | — | — | 0.053 |
| Dibasic Na Phosphate | 0.070 | — | — | — | — | — | — | 0.020 |
| Methylparaben | | | 0.020 | | | 0.020 | — | — |
| Propylparaben | | | 0.005 | | | 0.005 | — | — |
| Colorant⁴ | 0.020 | — | 0.010 | 0.020 | 0.020 | — | — | 0.020 |
| Citric Acid | | | | | | | 0.052 | 0.052 |
| Na Citrate | | | | | | | 0.212 | 0.212 |
| Ethanol | — | — | — | 1.200 | 5.000 | — | — | 5.000 |
| Water, Purified | QS | QS | QS | QS | QS | QS | QS | QS |

¹35% Solution Cosmetic Grade Hydrogen Peroxide
²Polyphosphate with average chain length from 18–30 supplied by ICL Performance Products
³Sweetener is sodium saccharin, sucralose or mixtures thereof.
⁴Colorant may include plaque-disclosing agent.

### Antimicrobial Dentifrice Compositions

| Component | Ex. 1 | Ex. 2 | Ex. 3 | Ex. 4 | Ex. 5 | Ex. 6 |
|---|---|---|---|---|---|---|
| Stannous Fluoride | 0.454 | 0.454 | | | — | 0.454 |
| Stannous Chloride | | | 1.500 | | | 1.500 |
| Zinc Lactate Dihydrate | 2.500 | | | | — | |
| Zinc Citrate Dihydrate | — | | | | 2.000 | |
| Zinc Carbonate¹ | | 1.000 | 2.000 | | | |
| Triclosan | — | | | 0.280 | — | |
| Na polyphosphate² | 13.000 | | | 7.000 | 14.030 | |
| Sodium Fluoride | — | | 0.243 | 0.243 | 0.243 | |
| Phytic Acid (20% Soln) | | 2.000 | | | | |
| Sodium Gluconate | 0.652 | 0.600 | 0.672 | | 0.650 | 2.100 |
| Glycerin | 38.519 | | | 57.725 | 38.400 | 14.425 |
| Sorbitol Soln. | | 35.785 | 34.275 | | | 37.496 |
| PEG-300 | 7.000 | | | 5.000 | 7.000 | |
| Propylene Glycol | 7.000 | | | | 7.000 | |
| Carboxymethylcellulose (CMC) | | 1.200 | 1.200 | | | 0.600 |
| Hydroxyethylcelluose (HEC) | | 0.300 | 0.300 | | | |
| Carageenan | 0.600 | 0.500 | 0.500 | 0.900 | 0.600 | |
| Xanthan Gum | 0.350 | | | | 0.350 | 0.700 |
| Silica abrasive | 25.000 | 16.000 | 20.000 | 20.000 | 25.000 | 20.000 |
| Titanium Dioxide | | 0.525 | 0.525 | | | 0.525 |
| Sodium Lauryl Sulfate (28% soln) | 2.500 | 7.500 | 7.500 | 6.000 | 2.500 | 5.000 |
| Sodium Lauryl Sulfate, powdered | — | | | | — | |
| Betaine | — | | | 1.500 | — | |
| Flavor | 0.800 | 0.950 | 0.950 | 1.100 | 0.600 | 1.000 |
| Sodium Saccharin | 0.500 | 0.250 | 0.250 | 0.250 | 0.500 | 0.300 |
| Trisodium Phosphate | 1.100 | | | | — | |
| Sodium Hydroxide | — | 0.122 | 0.006 | | — | 0.600 |
| Water and Minors, e.g., Speckles, | QS | QS | QS | QS | QS | QS |
| Colorant³ | | | | | | |

¹Zinc Carbonate AC supplied by Bruggemann Chemical: Newtown Square, PA, USA
²Polyphosphate with average chain length from 18–30 supplied by ICL Performance Products
³Colorant may include plaque-disclosing agent.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral hygiene regimen for achieving optimum oral health and hygiene, comprising at least once daily of each of the following steps:
(a) brushing teeth and oral cavity surfaces with an antimicrobial fluoride dentifrice, and
(b) rinsing the oral cavity with an antimicrobial mouthrinse,
wherein at least one of steps a) and b) is conducted prior to retiring at night and wherein the regimen provides effective antimicrobial activity in the mouth and benefits of reducing or inhibiting one or more of plaque, plaque regrowth, caries, calculus, gingivitis and breath malodor.

2. A regimen according to claim 1, further comprising as step (c) treating interproximal, gingival and subgingival areas using an interproximal device selected from dental floss, floss pick, dental tape and proxy brush, wherein step (c) is conducted immediately before or immediately after one or both of steps (a) and (b).

3. A regimen according to claim 2, wherein the interproximal device incorporates an antimicrobial agent.

4. A regimen according to claim 2, wherein each of steps (a) to (c) is conducted at least twice daily.

5. A regimen according to claim 4, wherein steps (a) and (b) are spaced for at least about 30 minutes.

6. A regimen according to claim 4, wherein at least one of step (a) or step (b) is conducted after meals.

7. A regimen according to claim 1 wherein a power toothbrush is used for brushing.

8. A regimen according to claim 1 wherein step (a) comprises brushing teeth and oral cavity surfaces with a dentifrice comprising
(i) a stannous ion source in an effective antimicrobial amount,
(ii) a fluoride ion source in an effective anticaries amount, and
(iii) one or more linear polyphosphates having an average chain length of about 4 or more.

9. A regimen according to claim 1 wherein step (b) comprises rinsing teeth and oral cavity surfaces with a mouthrinse comprising
(i) one or a mixture of quaternary ammonium antimicrobial agents in an amount to deliver at least about 300 ppm bioavailable quaternary ammonium antimicrobial agent selected from the group consisting of cetylpyridinium chloride, tetradecylpyridinium chloride, N-tetradecyl-4-ethyl pyridinium chloride, domiphen bromide, and mixtures thereof, and
(ii) a pharmaceutically-acceptable liquid carrier comprising a major proportion of water and from about 5% to about 30% by weight of the composition of a polyhydric alcohol humectant.

10. A regimen according to claim 9 wherein the mouthrinse comprises at least about 0.035% cetylpyridinium chloride by weight of the composition.

11. A regimen according to claim 1, wherein either dentifrice or mouthrinse comprises a plaque disclosing agent to identify areas with plaque buildup.

12. An oral hygiene kit comprising an antimicrobial fluoride dentifrice, a toothbrush, an antimicrobial mouthrinse, an interproximal device and instructions for a daily regimen comprising at least once daily of each of the following steps:
(a) brushing teeth and oral cavity surfaces with the dentifrice, and
(b) rinsing the oral cavity with the mouthrinse,
wherein at least one of steps (a) and (b) is conducted prior to retiring at night, wherein the toothpaste comprises
(i) a safe and therapeutically effective amount of an antimicrobial agent selected from a stannous ion source, a zinc ion source, triclosan, a peroxide source, cetyl pyridinium chloride, chlorhexidine, triclosan, triclosan monophosphate, essential oils and mixtures thereof,
(ii) a safe and effective amount of a fluoride ion source,
(iii) an abrasive; and
wherein the mouthrinse comprises
(i) a safe and therapeutically effective amount of an antimicrobial agent selected a stannous ion source, a zinc ion source, triclosan, a peroxide source, cetyl pyridinium chloride, domiphen bromide, chlorhexidine, triclosan, triclosan monophosphate, essential oils and mixtures thereof; and
(ii) an aqueous liquid carrier.

13. An oral hygiene kit according to claim 12, further comprising a plaque disclosing agent to identify areas with plaque buildup.

14. An oral hygiene kit according to claim 13, wherein the plaque disclosing agent is a component of any one of the dentifrice, mouthrinse or interproximal device or is a component of a separate oral care product included in the kit.

15. An oral hygiene kit according to claim 12, wherein the toothbrush is a power toothbrush.

16. An oral hygiene kit according to claim 12 including instructions to conduct at least one of step (a) or step (b) after meals.
